(19) United States
(12) Patent Application Publication
(10) Pub. No.: US 2015/0201883 A1
(43) Pub. Date: Jul. 23, 2015

(54) INTRINSIC FREQUENCY BASED DETERMINATION OF INSULIN RESISTANCE

(71) Applicant: CALIFORNIA INSTITUTE OF TECHNOLOGY, Pasadena, CA (US)

(72) Inventors: Danny Petrasek, Los Angeles, CA (US); Niema Pahlevan, Pasadena, CA (US); Morteza Gharib, Altadena, CA (US)

(21) Appl. No.: 14/602,133

(22) Filed: Jan. 21, 2015

Related U.S. Application Data
(60) Provisional application No. 62/101,466, filed on Jan. 9, 2015.
(60) Provisional application No. 61/930,389, filed on Jan. 22, 2014.

(51) Int. Cl. A61B 5/00; A61B 5/024

(52) U.S. Cl. CPC A61B 5/4866; A61B 5/024; A61B 5/726

(57) ABSTRACT

Systems, devices, and methods are described in which Intrinsic Frequency calculation (direct and/or by approximation) of a pulse pressure waveform is used make a determination of metabolic syndrome and/or insulin resistance or sensitivity. The pulse pressure waveform may be obtained non-invasively using a smartphone platform or be otherwise obtained.

### RELATED APPLICATIONS

This application claims the benefit of and priority to U.S. Provisional Patent Application Ser. No. 61/930,389, filed Jan. 22, 2014, and Ser. No. 62/101,466, filed Jan. 9, 2015, both of which are incorporated by reference in their entirety and for all purposes.

### FIELD

The embodiments described herein relate generally to Intrinsic Frequency (IF) calculation (direct and/or by approximation) of a pulse pressure waveform and insulin resistance and/or metabolic syndrome determinations therefrom.

### BACKGROUND

Metabolic syndrome is a highly prevalent clinical condition that by some estimates affects 34% of the adult population in North America. It is characterized by a constellation of hypertension, central obesity, glucose intolerance and hyperlipidemia. The underlying pathophysiology of metabolic syndrome is considered to be insulin resistance. However, the resistance can occur before any of the other features of the syndrome appear. Insulin resistance is believed to induce damage in the cardiovascular system, glucose and lipid metabolism as well and other tissue targets.

Notable among the effects of insulin resistance is an increase in aortic rigidity. The mechanism by which this occurs has been studied and is ongoing. It has been established by several key studies that Hyperinsulinemia in Diabetes type II (DM2) injures the structure of the arterial walls by altering (decreasing) the effective elastin/collagen ratio resulting in stiffer and less compliant blood vessels.

Measuring insulin resistance is challenging in practice. The so-called “gold standard” for such measurement is performed by an invasive and time consuming procedure called a glucose clamp study. A glucose clamp study is conducted in a hospital or clinical research facility and is decidedly impractical for routine clinical use. Currently there is no simple clinical technique that can measure insulin resistance so that on-going clinical evaluation and medical therapy can be adjusted.

Given the seriousness of conditions accompanying metabolic syndrome or caused by insulin resistance, a need exists for testing that is practical. Ideally, such testing is suitable for routine clinical use, or possibly even home or self-administer use. Embodiments described below meet this need or these needs and others that may be further apparent after review of the subject specification by those with skill in the art.

### SUMMARY

Provided herein are numerous example embodiments of systems (including sensor hardware referenced herein and the addition of a computer processor and other ancillary/support electronics and/or various housing elements), device components or sub-components of such systems, methods (including software programming and/or associated hardware for carrying out specified acts) and user interface (UI) features (including layouts and options and/or methodology associated with system use). Many of the embodiments may be adapted for wearable as well as hand-held use.

Various systems, devices, methods, features and advantages of the subject matter described herein will be or will become apparent to one with skill in the art upon examination of the following figures and Detailed Description. It is intended that all such systems, devices, methods, features and advantages be included within this description, be within the scope of the subject matter described herein and be protected or protectable by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are diagrams illustrating dynamic coupling of the heart and aorta in a human circulatory system.

FIG. 2A is a perspective view depicting pulse waveform acquisition from a subject using an example embodiment of a smart phone system.

FIG. 2B is an overview of an example embodiment of a pulse acquisition system.

FIG. 3 is a flowchart depicting an example embodiment of a method of using a pulse acquisition system.

FIGS. 4A, 5A, 6A and 7A are graphs depicting example pulse pressure waveforms and FIGS. 4B, 5B, 6B and 7B are graphs depicting example associated instantaneous and intrinsic frequency waveform calculations.

FIG. 8 is a graph depicting an example correlation between Δω and PWV.

FIGS. 9A and 9B are graphs depicting example Δω values plotted against age for different pulse pressure waveform acquisition sites.

FIGS. 10A and 10B are graphs depicting example correlations between insulin resistance (via an insulin sensitivty index) with PWV and Δω, respectively.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

It should be noted that all features, elements, components, functions, acts and steps described with respect to any embodiment provided herein are intended to be freely combinable and substitutable with those from any other embodiment. If a certain feature, element, component, function, act or step is described with respect to only one embodiment, then it should be understood that that feature, element, component, function, act or step can be used with every other embodiment described herein unless explicitly stated otherwise. This paragraph therefore serves as antecedent basis and written support for the introduction of claims, at any time, that combine features, elements, components, functions, acts and steps from different embodiments, or that substitute features, elements, components, functions, acts and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

Various example embodiments are described below. Reference is made to these examples in a non-limiting sense. They are provided to illustrate more broadly applicable aspects of inventive aspects. Various changes may be made to the embodiments described and equivalents may be substituted without departing from their true spirit and scope. In addition, many modifications may be made to adapt a particular situation, material, composition of matter, process, process act(s) or step(s) to the objective(s), spirit or scope of the claims made herein.

That said, as pertinent to certain measurement and calculations performed in connection with the example embodiments, pressure and flow waves generated by the heart propagate in the compliant arterial vasculature. These waves are reflected at various reflection sites in the arterial system. The waves carry information about the heart, vascular system and coupling of heart and vasculature.

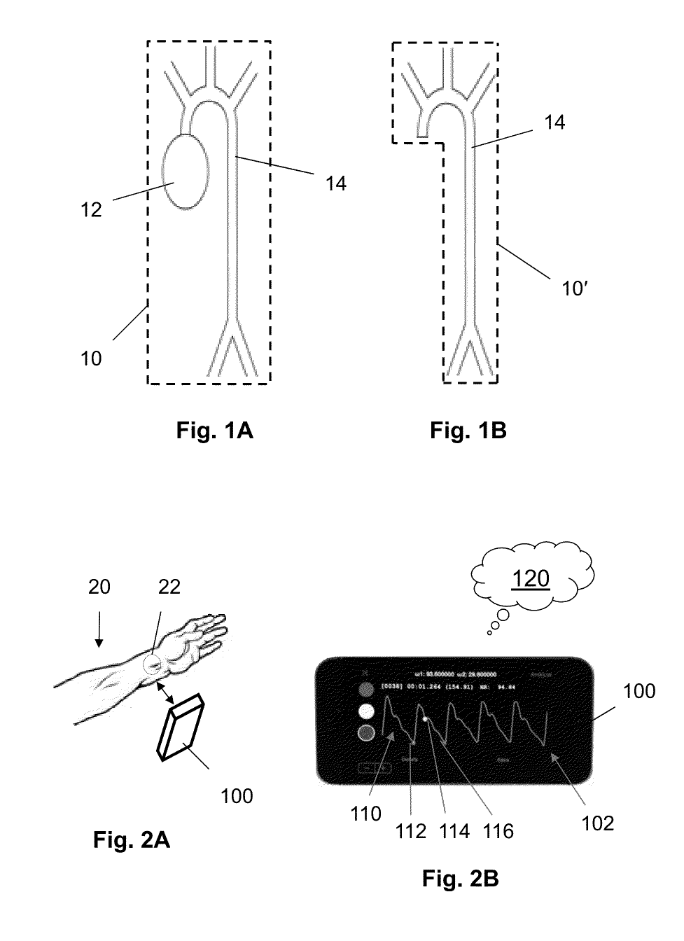

FIG. 1A illustrates a coupled heart-aorta system 10 in Systole (“S” phase) with the aortic valve open (not shown) and blood being pumped by the heart 12 into the aorta 14. The heart and aorta construct a coupled dynamic system before the closure of the aortic valve. As shown in FIG. 1B, after aortic valve closure during Diastole (“D” phase), the heart and aorta systems are decoupled in a second system state 10′. The aortic waves (i.e., pulse pressure or hemodynamic waves) in each state include information about heart dynamics, arterial network dynamic and heart-aorta coupling.

Intrinsic Frequency (IF) calculation of a pulse pressure waveform (e.g., as described in US Publ. No. 2013/0184573, which is incorporated by reference herein in its entirety and for all purposes) can be applied as described herein to determine insulin resistance and/or sensitivity. Intrinsic Frequency (IF) calculations as previously described can be applied to evaluate the cardiovascular system by portioning the system into a connected heart-aorta state 10 and a disconnected 10′ heart-aorta state as shown in FIGS. 1A and 1B.

With IF analysis, the characteristic or dominant frequency of each state is calculated and the results are given as ω₁ and ω₂, respectively. The ω₂ index is physiologically related to the vasculature and strongly correlates to pulse wave velocity and aortic rigidity. A rising ω₂ or decreasing Δω (i.e., ω₁, ω₂) given by various algorithms is consistent with the pathological states of aortic rigidity, insulin resistance, and others (e.g., as in metabolic syndrome).

Notably, as described in the US Publ. No. 2013/0184573 publication, IF methodology is a simplified and modified version of Sparse Time-Frequency Representation (STFR). The general STFR problem is defined as follows:

$$Minimize M \\ Subject to \text{:} s ( t ) = {\sum }_{i = 1}^{M} {a}_{i} ( t ) cos {θ}_{i} ( t ) , {a}_{i} ( t ) cos {θ}_{i} ( t ) ∈ D \\ i = 1 , … , M \qquad ( 1 )$$

In the example method(s), a simplified and modified version of STFR may be employed by minimizing:

$${‖ \begin{matrix}f ( t ) - {a}_{1} X ( 0 , {T}_{0} ) Cos {ω}_{1} t - {b}_{1} X ( 0 , {T}_{0} ) Sin {ω}_{1} t - \\ {a}_{2} X ( {T}_{0} , T ) Cos {ω}_{2} t - {b}_{2} X ( {T}_{0} , T ) Sin {ω}_{2} t - c\end{matrix} ‖}_{2}^{2} \\ X ( a , b ) = \{ \begin{matrix}1 & a \leq t \leq b \\ 0 & otherwise\end{matrix} \\ Subject to \text{:} \\ \{ \begin{matrix}{a}_{1} Cos {ω}_{1} {T}_{0} + {b}_{1} Sin {ω}_{1} {T}_{0} = {a}_{2} Cos {ω}_{2} {T}_{0} + {b}_{2} Sin {ω}_{2} {T}_{0} \\ {a}_{1} = {a}_{2} Cos {ω}_{2} T + {b}_{2} Sin {ω}_{2} T\end{matrix} \qquad ( 2 )$$

where, T₀ is the time of aortic valve closure (i.e., at the Dicrotic Notch) in order to determine intrinsic/dominant frequency (i.e., IF) values (ω₁, ω₂) in the two domains on either side of the Dicrotic Notch.

Still, it is to be recognized that the IF values (ω₁, ω₂) can be approximated and still fall within the spirit and scope of the embodiments hereof. In one example, the IF values are approximated using the graph of the instantaneous frequency (\dot{θ}₁(t)) of method of equation (1).

Other indices that can be used to approximate ω₁ and ω₂ as such include:
ω̅₁̅ approximating ω₁ by averaging the \dot{θ}₁(t) over an specific time period before the \dot{θ}₁(t) transition (when aortic valve is open);
ω̅₂̅ approximating ω₂ by averaging the \dot{θ}₁(t) over an specific time period after the \dot{θ}₁(t) transition (when aortic valve is closed);
\hat{ω}\hat{ω₁} approximating ω₁ by averaging the maximum and minimum value of \dot{θ}₁(t) curve before the \dot{θ}₁(t) transition (when aortic valve is open);
\hat{ω}\hat{ω₂} approximating ω₂ by averaging the maximum and minimum value of \dot{θ}₁(t) curve after the \dot{θ}₁(t) transition (when aortic valve is closed);
ω₁^{max} approximating ω₁ using the one of the local maximum of \dot{θ}₁(t) curve before the \dot{θ}₁(t) transition (when aortic valve is open);
ω₁^{min} approximating ω₁ using the one of the local minimum of \dot{θ}₁(t) curve before the \dot{θ}₁(t) transition (when aortic valve is open);
ω₂^{max} approximating ω₁ using the one of the local maximum of \dot{θ}₁(t) curve after the \dot{θ}₁(t) transition (when aortic valve is closed); and
ω₂^{min} approximating ω₁ using the one of the local minimum of \dot{θ}₁(t) curve after the \dot{θ}₁(t) transition (when aortic valve is closed).
   Likewise, it is possible to calculate precise or approximated IF values by other known time-frequency analyses such as Empirical Mode Decomposition (EMD) methods (see, e.g., U.S. Pat. No. 6,738,734 to Huang, incorporated herein by reference in its entirety and for all purposes) and Wavelet methods. In any case, use of the terms ω₁, ω₂ and Δω as applied herein are intended to encompass direct IF calculation as well as approximations thereof using such other methodology as exemplified above.

More generally, Pulse Wave Velocity (PWV) is a cardiovascular system metric that has been useful in analyzing the mechanical structure of the vasculature. It has been observed that when aortic rigidity is present, PWV is markedly greater. Consistent with this observation is that PWV increases with aging, as does pulse pressure. It has also been shown that PWV is elevated in subjects with Type II Diabetics (DM2). DM2 is the dominant form of DM making up over 90% of all diabetics in the world. DM2 is a disease of hyperinsulinemia and insulin resistance. In addition to diabetes, there are also other diseases with an underlying pathology is insulin resistance: including polycystic ovary disease, acanthosis nigracans and cortico steroid dependent patients. The pulse wave velocity in DM2 is increased given the effect of hyperinsulinemia to the vascular remodeling of elastin and collagen.

However, PWV is strictly a one-dimensional measure of the speed of the forward signal that emanates from the heart and travels down an arterial path. The choice of a particular path or paths may yield insight into the local physiology as compared to other paths (i.e. Carotid-femoral vs. others). In contrast, the pulse pressure curve contains signals that include the forward signal, the reflected signal as well as the local physiology. ω₂ represents the vascular or D phase (see FIG. 1B) of the cycle described above. Δω includes both the S (see FIG. 1A) and D phases and perhaps a more information regarding the vascular system. Accordingly, the subject IF methods may provide a deeper insight into the physiology of insulin resistance and vascular dynamics compared with PWV.

Notably, one of the criticisms and challenges of using PWV is that measurement is highly dependent on the assumed vessel length. In use, the length under most circumstances is an estimated length.

The IF methodologies of the present embodiments do not need to rely on vessel length for their measurements. Rather the analysis can depend strictly on the pulse pressure signal regardless of the path length. Moreover, the information contained in the signal maybe different depending on which signal acquisition site is being measured. This creates an opportunity to learn more about the cardiovascular system and the regional physiology.

IF methodology presents an opportunity to monitor or make a diagnosis or determination regarding metabolic syndrome and/or insulin resistance with the ease of use associated with a “smart phone” or other hand-held device. The ease of diagnosis and option of subsequent treatment could easily lead to an enormous impact on health and health care costs. The use of an insulin resistance test could become as common and routine as measuring pulse. The methodology can be an integral component to managing clinically difficult patients in the ICU (intensive care unit) and insulin pump dependent diabetics.

As referenced above, insulin resistance occurs is a precursor to the actual clinical detection of the disease itself (hypertension, DM2). The advantage of having this diagnostic tool on a mobile phone creates access that is global. Beyond the clinical advantages, the methodology may give new insight into the evolution of the pathology from childhood to old age.

In one embodiment, the pulse pressure waveform is obtained non-invasively such as by using mobile phone. As indicated by the double arrow in FIG. 2A, this may be accomplished using the mobile phone 100 camera employed as a sensor for light reflected from the device LED flash from the skin of as subject 20. In this example, the pressure waveform is being obtained at a/the radial location 22. U.S. Pat. No. 5,363,855, incorporated herein by reference in its entirety, details the manner in which such light-based tonometers may operate. Further optional approaches are shown and described in U.S. patent application Ser. No. 14/601,170, which is incorporated by reference herein in its entirety and for all purposes. As such, the display 102 of the smartphone 100 may be used as an interface for pulse waveform 110 feature identification (see dots 112, 114, 116), waveform, calculated IF parameter and/or diagnosis or result(s) display.

In which case, embodiments hereof provide a non-invasive system and/or methods for determining and/or quantifying the presence or degree of metabolic syndrome and/or insulin resistance in a subject, particularly (though not necessarily) human subjects. The Intrinsic Frequency (IF)—or associated—computation may be performed on the smartphone by its included processor(s). However, word-wide-web or so-called cloud 120 based computing may alternatively be employed. Such optional features or details are further described in the above-referenced '573 publication which is incorporated herein by reference.

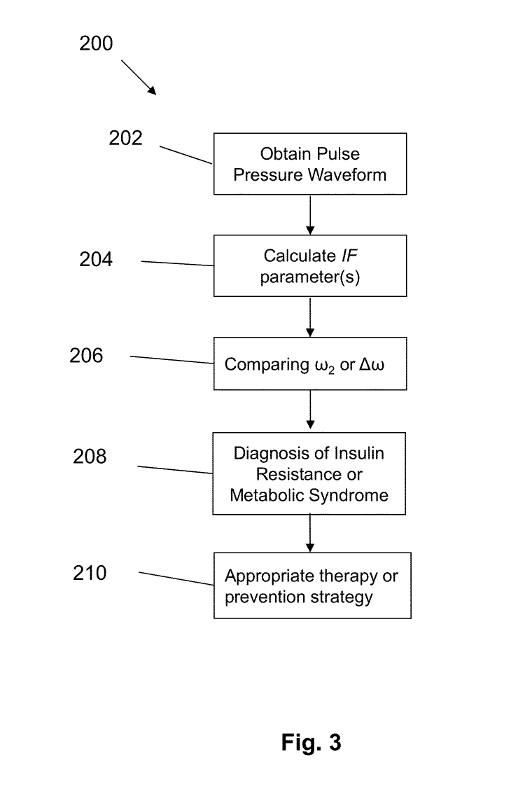

However, the pulse pressure waveform is obtained, methods and software running hardware according to such methodology may be carried out as detailed in FIG. 3. Here, a method or process 200 begins with obtaining one or more pulse pressure waveforms at 202. Per above, the pulse pressure waveform(s) may be taken for a subject on site and processing occur in real time. By “real time” what is meant is instantaneously or near-instantaneously—certainly, beyond any human computational capacity given the complexity of the IF calculations requiring computer processing. Alternatively, while the IF calculations may be performed by a computer instantaneously or near instantaneously, the pulse pressure waveform to be evaluated may be obtained in advance. It may even represent data stored previously in analog (e.g., printout) form that is digitized and then analyzed.

In any case, at 204 the pulse pressure waveform information or data is subject to IF calculation. At least an ω2 parameter (i.e, corresponding to the D phase of the cardiac cycle after the Dicrotic Notch) will be calculated a given pulse pressure waveform. In embodiments hereof, both ω1 (i.e., corresponding to the S phase of the cardiac cycle before the Dicrotic Notch) and ω2 are calculated. Further parsing of the pulse pressure waveform signal is possible as well. At 204, a comparison is made with ω2 and/or Δω. In this comparison, a high or higher or raising Δω or lower or lower or decreasing ω2 as calculated is indicative of metabolic syndrome and/or insulin resistance. This comparison may be as compared to a subject's previously obtained values (i.e., as in monitoring his or her condition over a period ranging from months to years) and/or with otherwise comparable “healthy” subject (for a given age) or other baseline value(s). The result of the comparison is shown at 206 in terms of a determination or diagnosis of insulin resistance and/or more generally of metabolic syndrome. Finally, such a diagnosis may be followed by suggestion (e.g., by the system or by a physician viewing the diagnosis results) of an appropriate treatment or prevention regimen or therapy at 210.

### EXAMPLES

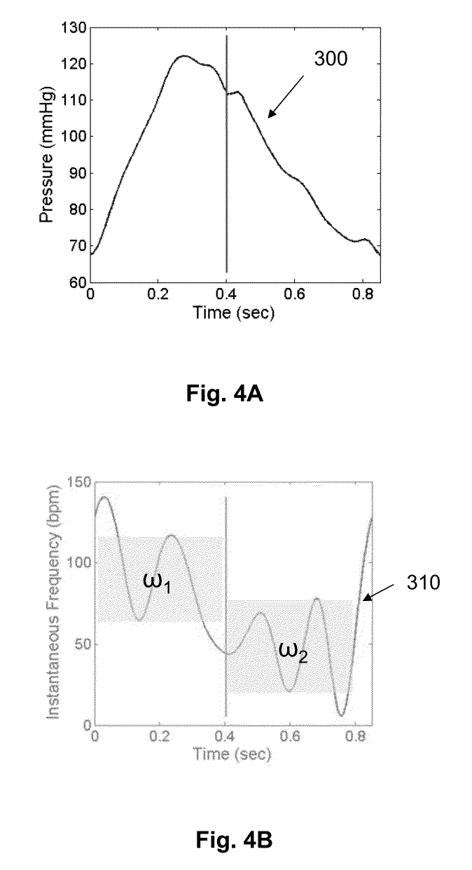

FIG. 4A illustrates a pulse pressure waveform 300 as may be used in embodiments hereof. The vertical line indicates the position of the Dicrotic Notch representing valve closure or change between the S phase shown in FIG. 1A (in which the Aortic valve is open and connect the heart to the arterial system) and the D phase (in which the Aortic valve is closed and the heart and the Arterial system are—in principle—disconnected). In FIG. 4B, the associated Intrinsic Frequency (IF) modes (ω₁, ω₂) of a calculated instantaneous frequency curve 310 are shown and highlighted in the shaded regions.

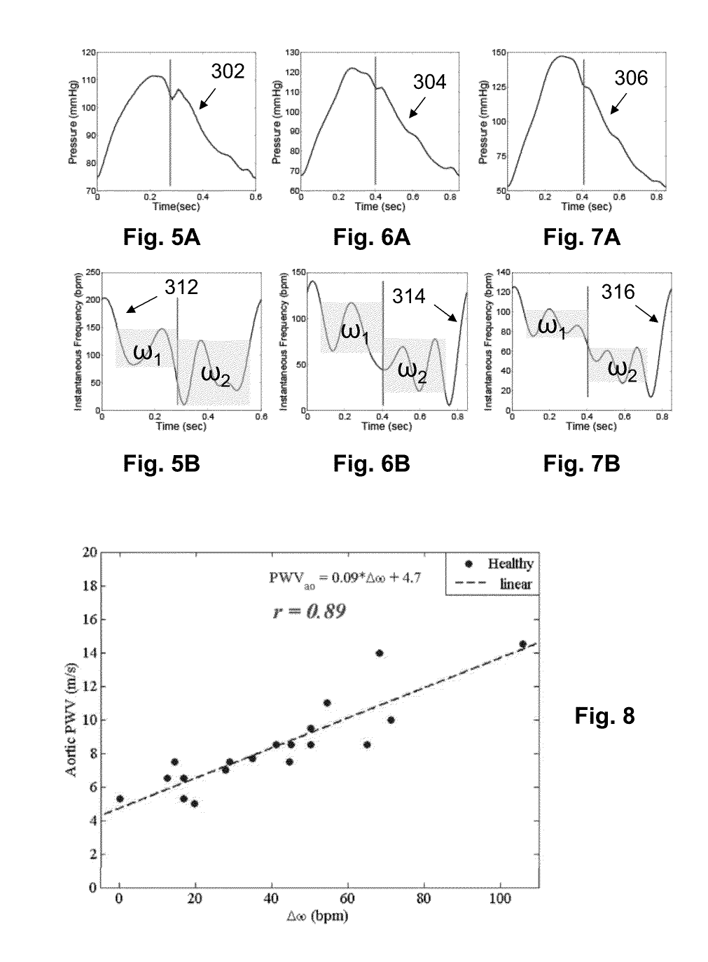

FIGS. 5A, 6A and 7A present additional pulse pressure waveform examples 302, 304, 306. FIGS. 5B, 6B and 7C show corresponding example intrinsic frequency curves 312, 314, 316 and IF modes (ω₂, ω₂) or ranges.

Notably, the height of the highlighted areas depicting ω₁ and ω₂ is similar in FIG. 5B. The Δω in this case is close (or closer as compared to other examples herein) to zero and thereby indicative as optimal with respect to the workload on the heart. This is the typical pattern seen with healthy young people (in this case a 30 year old individual). Increasing vascular rigidity (Elastance), results in the separation of the heights of the highlighted IF regions. Increase in Δω and (hence, rigidity) is shown in the example in FIGS. 6A-B and the highest rigidity and greatest Δω in the example of FIGS. 7A-B.

Likewise, PWV is known to increase with age. This increase is believed to be a result of increased rigidity in the central arteries. In FIG. 8, correlation is demonstrated between Δω and PWV. The Δω values were computed directly from waveforms obtained from healthy subjects and plotted against their corresponding PWV. The degree of correlation r=0.89 is significant.

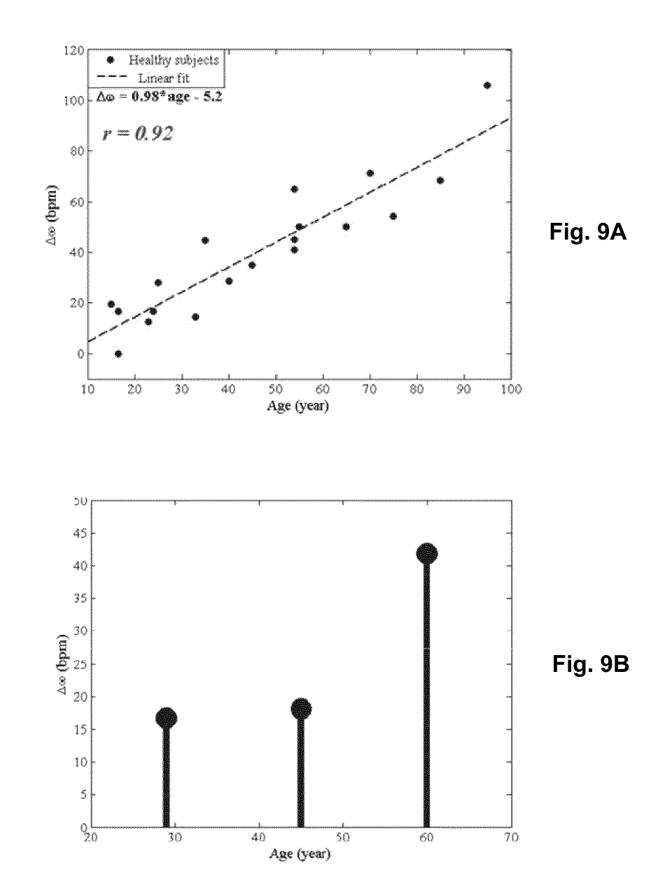

In FIG. 9A, computed Δω values are plotted against age. The pressure waveforms for the IF calculations for FIG. 9A were obtained from the carotid artery data and thus representing the central artery physiology. The value r=0.92 demonstrates the high degree of correlation in the data. FIG. 9B shows the effect of aging and Δω by computing the values from waveforms obtained by finger plethysmography. As compared to the data plotted in the previous chart, this chart (i.e., that of FIG. 9B) clearly shows a measurable effect in the peripheral arterial system where the influence of arterioles and muscular arteries are significant.

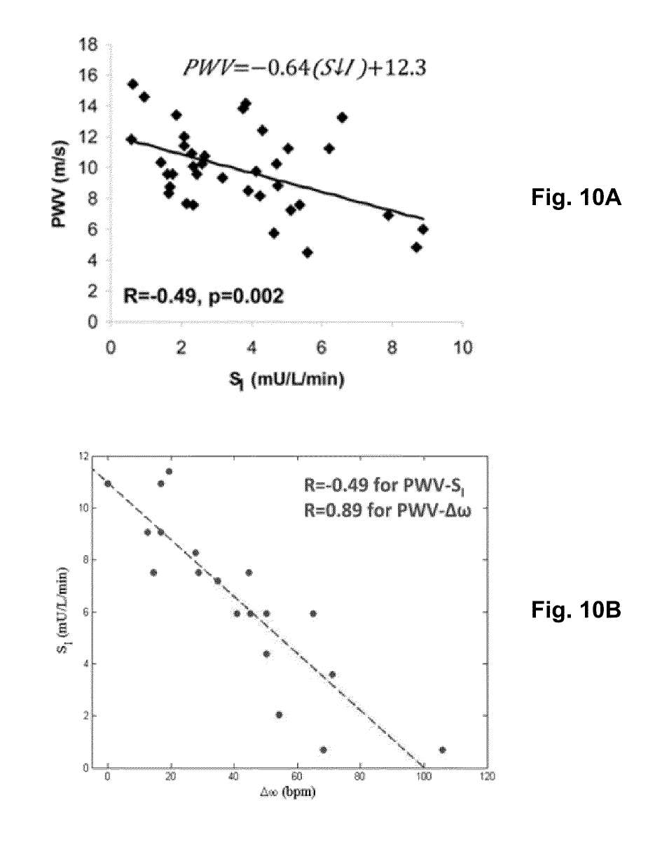

FIG. 10A illustrates the known correlation of insulin resistance with PWV. This graph (from Sengstock, et al. The Journal of Clinical Endocrinology & Metabolism, 90.50(2005): 2823-2827) plots PWV velocity versus insulin sensitivity (“S_{I}”) where low insulin sensitivity (S_{I}) indicates insulin resistance. The statistical significance was calculated in the study to be p<0.002. Based on this graph and the correlation represented in FIG. 7 (i.e., relating PWV to Δω), a hypothetical rendering of Δω versus insulin S_{I} was prepared as presented in FIG. 10B. Since PWV and Δω are physiologically related, the graph in FIG. 10B is expected to accurately predict the relationship between Δω and insulin resistance. In any case, it will offer a guide that may be further refined as employed by those with skill in the art making diagnoses or determinations as enabled herein.

Variations

In addition to the embodiments disclosed already, still more variations are within the scope of this description.

The various illustrative processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a digital camera of any type including those using CMOS, CCD or other digital image capture technology.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available non-transitory media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

To the extent the embodiments disclosed herein include or operate in association with memory, storage, and/or computer readable media, then that memory, storage, and/or computer readable media are intended to be non-transitory. Accordingly, to the extent that memory, storage, and/or computer readable media are covered by one or more claims, then that memory, storage, and/or computer readable media is only non-transitory.

Operations as described herein can be carried out on or over a website or network. The website can be operated on a server computer or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., such as those already described. The programs may also be run over a network, for example, with a server or other machine sending signals to the local machine, which allows the local machine to carry out the operations described herein.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A method of analyzing a pulse pressure waveform of a subject, the method comprising:
obtaining a pulse pressure waveform including a Dicrotic Notch;
with at least one computer processor connected to receive data for the pulse pressure waveform, calculating an intrinsic frequency value after the Dicrotic Notch (ω₂); and
based on the ω₂ value, making a determination of insulin resistance or metabolic syndrome status of the subject.

2. The method of claim 1, wherein the pulse pressure waveform is captured using a smartphone.

3. The method of claim 1, wherein an intrinsic frequency value (ω₁) is also calculated before the Dicrotic Notch.

4. The method of claim 3, wherein the determination of insulin resistance or metabolic syndrome status employs ω₁-ω₂(Δω).

5. The method of claim 4, wherein Δω about zero is indicative of no insulin resistance or metabolic syndrome.

6. The method of claim 4, wherein Δω is correlated to Insulin Sensitivity (S_{I}) for the determination.

7. The method of claim 4, wherein Δω of a subject is monitored to make the determination.

8. The method of claim 7, wherein the monitoring is over a period of years.

9. The method of claim 1, wherein ω₂ of a subject is monitored to make the determination.

10. The method of claim 9, wherein the monitoring is over a period of years.

11. The method of claim 3, where the intrinsic frequency values are calculated using Intrinsic Frequency (IF) analysis.

12. The method of claim 3, wherein the intrinsic frequency values are calculated using a time-frequency analysis for approximation.

13. The method of claim 12, wherein the time-frequency approximation is calculated using a method selected from Sparse Time-Frequency Representation (STFR), Empirical Mode Decomposition (EMD), and Wavelet methods.

14-39. (canceled)
